(19) United States
(12) Patent Application Publication
(10) Pub. No.: US 2020/0062681 A1
(43) Pub. Date: Feb. 27, 2020

(54) Novel compositions and methods for synthesizing deep eutectic solvents from lignin derived phenolic compounds

(71) Applicants: National Technology & Engineering Solutions of Sandia, LLC, Albuquerque, NM (US); The Regents of the University of California, Oakland, CA (US)

(72) Inventors: Seema Singh, Clarksburg, MD (US); Kwang Ho Kim, Walnut Creek, CA (US); Blake A. Simmons, San Francisco, CA (US)

(73) Assignees: National Technology & Engineering Solutions of Sandia, LLC, Albuquerque, NM (US); The Regents of the University of California, Oakland, CA (US)

(21) Appl. No.: 16/672,075

(22) Filed: Nov. 1, 2019

Related U.S. Application Data
(63) Continuation of application No. PCT/US2018/030540, filed on May 1, 2018.
(60) Provisional application No. 62/492,694, filed on May 1, 2017.

(51) Int. Cl. C07C 39/08; C07C 59/48; C07C 39/11; C07C 47/565

(52) U.S. Cl. CPC C07C 39/08; C07C 47/565; C07C 39/11; C07C 59/48

(57) ABSTRACT

The present invention provides for a method to produce a deep eutectic solvent (DES) comprising: (a) providing one or more lignin derived monomeric phenol, or a mixture thereof, in a solution, (b) introducing one or more hydrogen acceptors, or a mixture thereof, to the solution, and (c) heating the solution, such that steps (b) and (c) together result in the synthesis of a DES.

### RELATED PATENT APPLICATIONS

The application claims priority as a continuation application to PCT International Patent Application No. PCT/US2018/030540, filed May 1, 2018, which claims priority to U.S. Provisional Patent Application Ser. No. 62/492,694, filed May 1, 2017, which are herein incorporated by reference in their entireties.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of synthesizing deep eutectic solvents.

### BACKGROUND OF THE INVENTION

Lignin is one of the most abundant biopolymers found in the planet earth. However, the residual lignin (20-30 wt % of the initial biomass) obtained after pretreatment is often overlooked as byproduct and in the paper and pulp industry and typically used for waste heat production. The structure of lignin suggests that it can be a valuable source of chemicals, particularly phenolics, which could significantly improve the economics of a biorefinery.

### SUMMARY OF THE INVENTION

The present invention provides for a method to produce a deep eutectic solvent (DES) comprising: (a) providing one or more lignin derived monomeric phenol, or a mixture thereof, in a solution, (b) introducing one or more hydrogen acceptors, or a mixture thereof, to the solution, and (c) heating the solution, such that steps (b) and (c) together result in the synthesis of a DES.

In some embodiments, the one or more lignin derived monomeric phenol comprises at least two, three, or four different lignin derived monomeric phenol compounds.

In some embodiments, the lignin derived monomeric phenol is any phenol with at least one or more additional substituents comprising an oxygen atom to the aromatic ring. The substituents comprising an oxygen atom can be a hydroxyl group, a carboxylic acid, an ether, alcohol, or an acyl.

In some embodiments, the lignin derived monomeric phenol is:

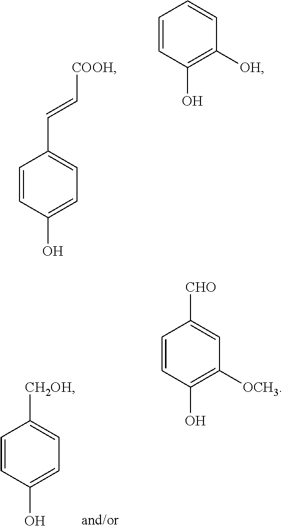 [SMILES: O=C(O)/C=C/c1ccc(O)cc1 ; Oc1ccccc1O ; OCc1ccc(O)cc1 ; COc1cc(C=O)ccc1O]

In some embodiments, the hydrogen acceptor is a quaternary ammonium salt comprising: (a) a quaternary ammonium cation comprising four alkyl groups covalently linked to the ammonium cation, wherein each of the four alkyl groups is independently selected from the group consisting of C₁-C₆ alkyl and at least one of the alkyl comprises a hydroxyl group as a substituent; and, (b) an anion selected from the group consisting of OH⁻, HSO₄⁻, H₂PO₄⁻, PO₄⁻, lysinate, HCO₃⁻, a carboxylic acid anion, a dicarboxylic acid anion, and Cl⁻. In some embodiments, the quaternary ammonium cation is a choline.

In some embodiments, the hydrogen acceptor is choline chloride (ChCl).

In some embodiments, the heating step comprises increasing the temperature of the solution to a value within a range of about 75° C. to about 125° C. In some embodiments, the heating step comprises increasing the temperature of the solution to a value within a range of about 80° C. to about 120° C. In some embodiments, the heating step comprises increasing the temperature of the solution to a value within a range of about 90° C. to about 110° C. In some embodiments, the heating step comprises increasing the temperature of the solution to about 100° C.

The present invention provides for a method of pretreating biomass comprising: (a) providing a biomass, (b) pretreating the biomass with a DES system to produce a sugar and a lignin, (c) optionally separating the sugar and the lignin, (d) depolymerizing and/or converting the lignin into one or more lignin derived monomeric phenol, or a mixture thereof, (e) providing the one or more lignin derived monomeric phenol, or a mixture thereof, in a solution, (f) introducing one or more hydrogen acceptors, or a mixture thereof, to the solution, (g) heating the solution, such that steps (f) and (g) together result in the synthesis of a DES, (h) optionally forming a DES system from the DES synthesized in step (g), and (i) optionally repeating steps (a) to (h) using the DES system formed in step (h) in the pretreating step (b).

The present invention provides for a DES system formed by a method described herein.

The present invention provides for novel compositions described herein.

Deep eutectic solvents (DESs) share the promising solvent properties of ionic liquids. They show low volatility, wide liquid range, water-compatibility, non-flammability, non-toxicity, biocompatibility and biodegradability. Furthermore, DES can be easily prepared from readily available materials at high purities and low cost compared to ILs. Lignin is the second most abundant naturally occurring polymer next to cellulose, which represents a significant component of carbon on earth. Large amount of technical lignins such as Kraft lignin and lignosulfonate is produced as by-products in the pulp and paper industries. It is also expected that more lignin will become available in coming years as the production capability of second generation of biofuels increases. As a renewable and resource, lignin and lignin derived products (phenolics) will be important material. In this invention, we propose novel DESs with lignin-derived phenolic compounds either as a single monomer or phenolic mixture with the goal of developing cheap, environmental benign, and renewable solvent system for biomass process.

Deep eutectic solvents (DESs) share the promising solvent properties of ionic liquids. They show low volatility, wide liquid range, water-compatibility, non-flammability, non-toxicity, biocompatibility and biodegradability. Furthermore, DES can be easily prepared from readily available materials at high purities and low cost compared to ILs. Lignin is the second most abundant naturally occurring polymer next to cellulose, which represents a significant component of carbon on earth. Large amount of technical lignins such as Kraft lignin and lignosulfonate is produced as by-products in the pulp and paper industries. It is also expected that more lignin will become available in coming years as the production capability of second generation of biofuels increases. As a renewable and resource, lignin and lignin derived products (phenolics) are important material. In this invention, we propose novel DESs with lignin-derived phenolic compounds either as a single monomer or phenolic mixture with the goal of developing cheap, environmental benign, and renewable solvent system for biomass process.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 shows the formation of DES by the heating of quaternary ammonium salts with a hydrogen donor.

FIG. 2 shows a schematic representation of a eutectic point on a two-component phase diagram.

FIG. 3 shows a synthesis of lignin derived DES from biomass.

FIG. 4 shows glucose yield from switchgrass biomass by pretreatments using different DES.

FIG. 5 shows a closed-loop biorefinery concept using DESs derived from lignin.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” includes any value up to 10% less or 10% more inclusive of the value the term describes.

The term “quaternary ammonium” refers to ammonium compounds of general formula ⁺N—(R)₄ wherein R is an alkyl or an aryl group, an exemplary quaternary ammonium compound is ChCl or tetrabutylammonium.

The term “lignin” also includes the meaning of a mixture of different lignin polymers.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

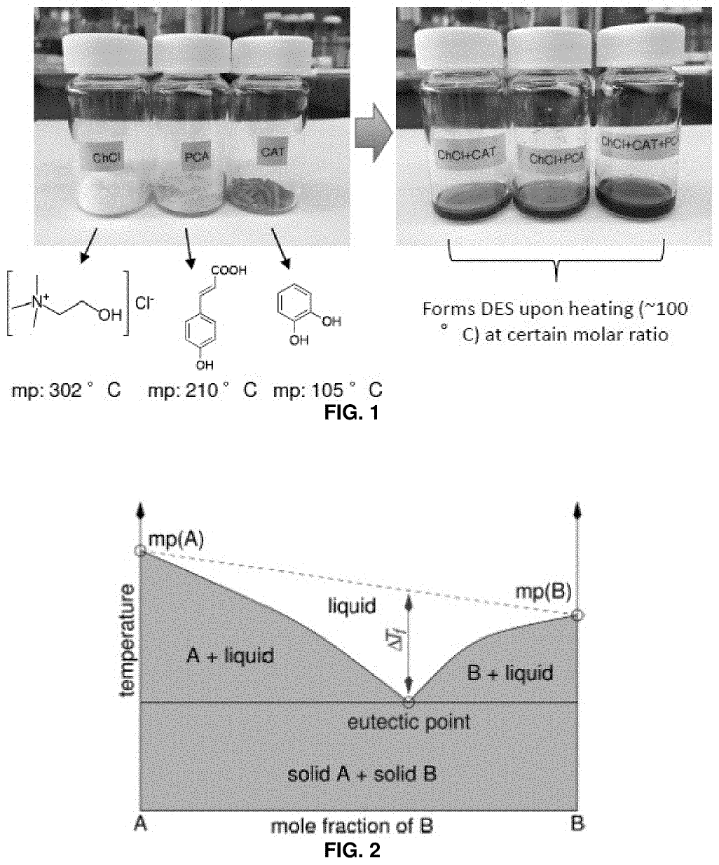

DESs are systems formed from a eutectic mixture of Lewis or Brønsted acids and bases which can contain a variety of anionic and/or cationic species. DESs can form a eutectic point in a two-component phase system (FIG. 2). DESs are formed by complexation of quaternary ammonium salts (such as, choline chloride) with hydrogen bond donors (HBD) such as amines, amides, alcohols, or carboxylic acids (FIG. 1). The interaction of the HBD with the quaternary salt reduces the anion-cation electrostatic force, thus decreasing the melting point of the mixture. DESs share many features of conventional ionic liquid (IL), and promising applications would be in biomass processing, electrochemistry, and the like.

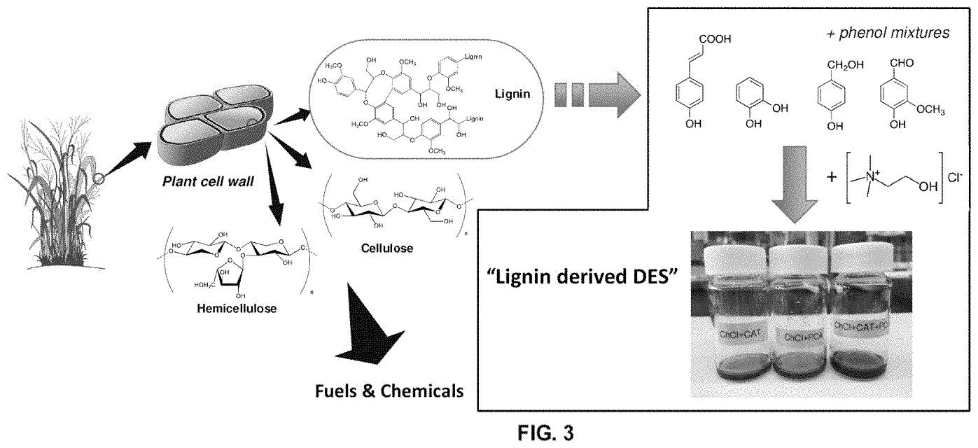

Typically, DES is prepared using an alcohol (such as glycerol or ethylene glycol), amines (such as urea), and an acid (such as oxalic acid or lactic acid). The present invention provides a new class of renewable DESs with lignin-derived phenols as HBDs (FIG. 3). Results demonstrate that both phenolic monomers and phenol mixture readily form DES upon heating at 100° C. with specific molar ratio with choline chloride. This new class of DES does not require a multistep synthesis. The novel DES is synthesized from lignin which is a renewable source.

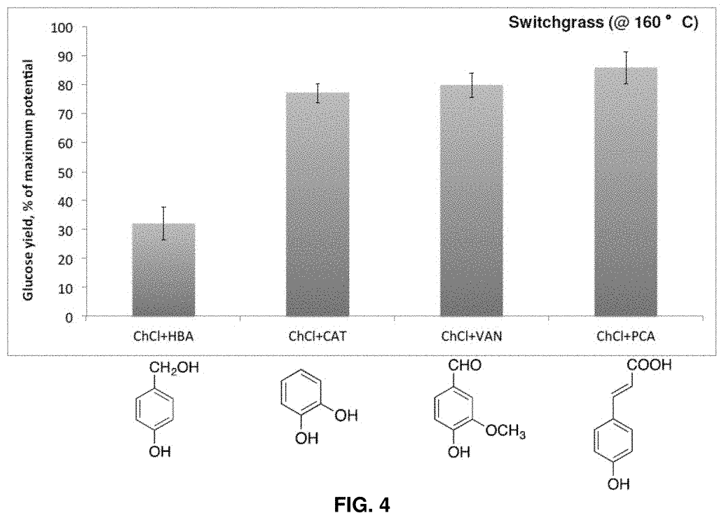

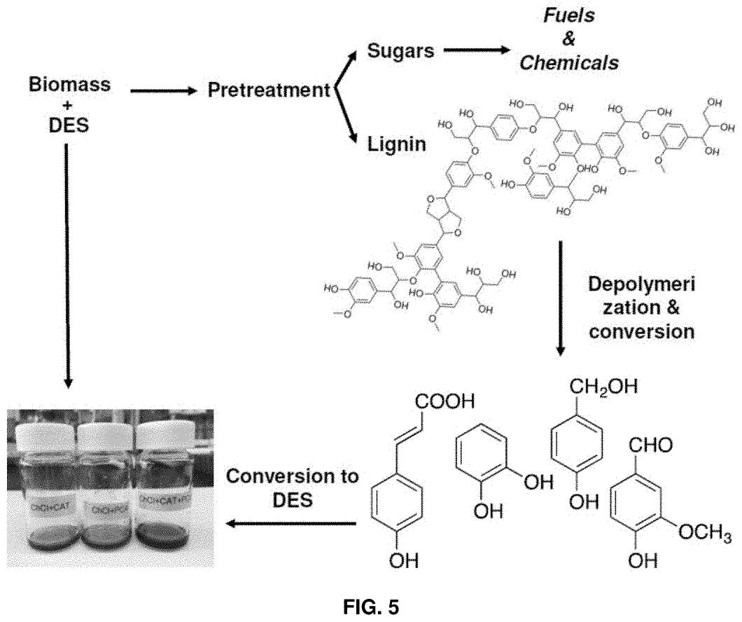

Both monomeric phenols and phenol mixture can be used to prepare DES. DES is capable of dissolving biomass or lignin, and can be utilized in biomass pretreatment and other applications. Using DES produced from biomass could lower the cost of biomass processing and enable greener routes for a variety of industrially relevant processes. See FIGS. 4 and 5.

Deep eutectic solvents (DESs) are widely acknowledged as a new class of ionic liquid. DES contains large, nonsymmetrical ions that have low lattice energy and hence low melting points. DES is a mixture of two or more chemicals acting as either hydrogen bond donors or acceptors. It has been gaining attention because DES is capable of dissolving biomass or lignin. DES can provide a mild acid-base catalysis mechanisms which allows the controlled cleavage of labile linkages in biomass cell wall structure, thus leading to lignin depolymerization and separation from the biomass. Herein is described the formation of novel DESs prepared by lignin-derived phenols, which are renewable sources obtained from lignocellulosic biomass. In a particular embodiment, DES is synthesized using lignin-derived phenols, choline chloride (ChCl), as a hydrogen acceptor and several phenolic compounds including catechol (CAT), vanillin (VAN), 4-hydroxybenzylalcohol (HBA) and p-coumaric acid (PCA) as hydrogen bond donors. The mixture of ChCl and each phenol/phenol mixture with certain molar ratio forms DES after heat treatment at about 100° C. The novel DES synthesized with lignin-derived phenols can be used as solvent for biomass pretreatment, lignin depolymerization, etc., achieving whole biomass utilization. One aspect of this invention is the synthesis of novel and renewable DESs from lignin. Several DESs have been synthesized using monomeric phenols with goal of finding environmental benign solvents for biomass processing. A preliminary result revealed that both phenolic monomers and phenolic mixture can readily form DES upon heating at about 100° C. with specific molar ratio with ChCl.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method to produce a deep eutectic solvent (DES) comprising: (a) providing one or more lignin derived monomeric phenol, or a mixture thereof, in a solution, (b) introducing one or more hydrogen acceptors, or a mixture thereof, to the solution, and (c) heating the solution, such that steps (b) and (c) together result in the synthesis of a DES.

2. The method of claim 1, wherein the lignin derived monomeric phenol is a phenol with at least one or more additional substituents comprising an oxygen atom to the aromatic ring.

3. The method of claim 2, wherein the lignin derived monomeric phenol is:

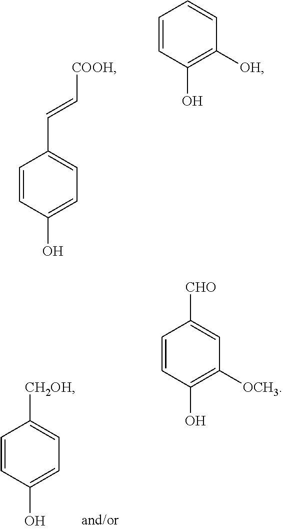 [SMILES: O=C(O)/C=C/c1ccc(O)cc1 ; Oc1ccccc1O ; OCc1ccc(O)cc1 ; COc1cc(C=O)ccc1O]

4. The method of claim 3, wherein the lignin derived monomeric phenol is:

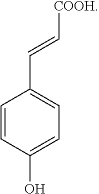 [SMILES: O=C(O)/C=C/c1ccc(O)cc1]

5. The method of claim 3, wherein the lignin derived monomeric phenol is:

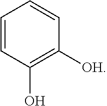

6. The method of claim 3, wherein the lignin derived monomeric phenol is:

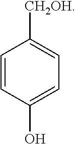

7. The method of claim 3, wherein the lignin derived monomeric phenol is:

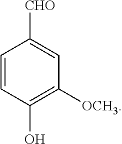 [SMILES: COc1cc(C=O)ccc1O]

8. The method of claim 1, wherein the hydrogen acceptor is a quaternary ammonium salt comprising: (a) a quaternary ammonium cation comprising four alkyl groups covalently linked to the ammonium cation, wherein each of the four alkyl groups is independently selected from the group consisting of C₁-C₆ alkyl and at least one of the alkyl comprises a hydroxyl group as a substituent; and, (b) an anion selected from the group consisting of OH⁻, HSO₄⁻, H₂PO₄⁻, PO₄⁻, lysinate, HCO₃⁻, a carboxylic acid anion, a dicarboxylic acid anion, and Cl⁻.

9. The method of claim 8, wherein the quaternary ammonium cation is a choline.

10. The method of claim 9, wherein the hydrogen acceptor is choline chloride (ChCl).

11. The method of claim 1, the heating step comprises increasing the temperature of the solution to a value within a range of about 75° C. to about 125° C.

12. A method of pretreating biomass comprising: (a) providing a biomass, (b) pretreating the biomass with a DES system to produce a sugar and a lignin, (c) optionally separating the sugar and the lignin, (d) depolymerizing and/or converting the lignin into one or more lignin derived monomeric phenol, or a mixture thereof, (e) providing the one or more lignin derived monomeric phenol, or a mixture thereof, in a solution, (f) introducing one or more quaternary ammonium salts, or a mixture thereof, to the solution, (g) heating the solution, such that steps (f) and (g) together result in the synthesis of a DES, (h) optionally forming a DES system from the DES synthesized in step (g), and (i) optionally repeating steps (a) to (h) using the DES system formed in step (h) in the pretreating step (b).

13. The method of claim 12, wherein the lignin derived monomeric phenol is a phenol with at least one or more additional substituents comprising an oxygen atom to the aromatic ring.

14. The method of claim 13, wherein the lignin derived monomeric phenol is:

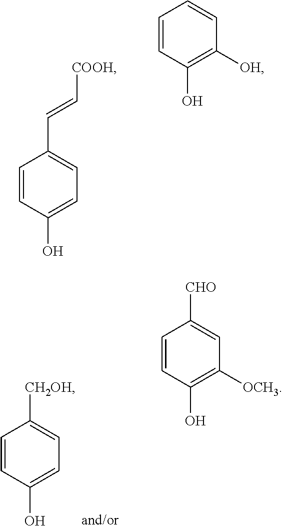 [SMILES: O=C(O)/C=C/c1ccc(O)cc1 ; Oc1ccccc1O ; OCc1ccc(O)cc1 ; COc1cc(C=O)ccc1O]

15. The method of claim 14, wherein the lignin derived monomeric phenol is:

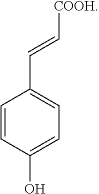 [SMILES: O=C(O)/C=C/c1ccc(O)cc1]

16. The method of claim 14, wherein the lignin derived monomeric phenol is:

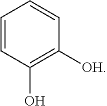

17. The method of claim 14, wherein the lignin derived monomeric phenol is:

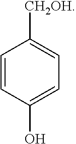

18. The method of claim 14, wherein the lignin derived monomeric phenol is:

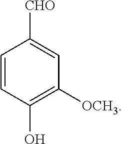 [SMILES: COc1cc(C=O)ccc1O]

19. The method of claim 12, wherein the hydrogen acceptor is a quaternary ammonium salt comprising: (a) a quaternary ammonium cation comprising four alkyl groups covalently linked to the ammonium cation, wherein each of the four alkyl groups is independently selected from the group consisting of C₁-C₆ alkyl and at least one of the alkyl comprises a hydroxyl group as a substituent; and, (b) an anion selected from the group consisting of OH⁻, HSO₄⁻, H₂PO₄⁻, PO₄⁻, lysinate, HCO₃⁻, a carboxylic acid anion, a dicarboxylic acid anion, and Cl⁻.

20. The method of claim 19, wherein the quaternary ammonium cation is a choline.

21. The method of claim 20, wherein the hydrogen acceptor is choline chloride (ChCl).

22. The method of claim 12, the (g) heating step comprises increasing the temperature of the solution to a value within a range of about 75° C. to about 125° C.
